(19) United States
(12) Patent Application Publication
(10) Pub. No.: US 2002/0115636 A1
(43) Pub. Date: Aug. 22, 2002

(54) Composition for treating equine gastric ulcers

(76) Inventor: J. Denzil Carpenter, St. Matthews, SC (US)

Correspondence Address: MICHAEL A. MANN, NEXSEN PRUET JACOBS & POLLARD LLC, PO DRWR 2426, COLUMBIA, SC, 29202-2426, US

(21) Appl. No.: 09/783,828

(22) Filed: Feb. 14, 2001

(51) Int. Cl. A61K 31/715; A61K 31/44

(52) U.S. Cl. 514/53; 514/161; 514/338; 514/492

(57) ABSTRACT

A composition for treating equine gastric ulcers comprises a mixture of a proton pump inhibitor, bismuth salicylate, and glycerin, preferably mixed with a sweetener and a flavoring agent. Preferably, the proton pump inhibitor is omeprazole. As an alternative to bismuth salicylate, bismuth citrate or sucralfate may be substituted. The ratio of acid antagonist to bismuth salicylate is approximately 1:3 by weight, and a sufficient quantity of glycerin is mixed with these to form a paste. The composition is given orally using a syringe in the amount of 15 ccs per day.

### FIELD OF THE INVENTION

The present invention relates generally to medicating large animals such as horses. In particular, it relates to a composition that includes an acid inhibitor for treating gastric ulcers in horses.

### BACKGROUND OF THE INVENTION

Gastric ulcers are a particular affliction of race horses. Over 9 out of 10 racehorses get ulcers sometime during their lives, many as foals. It has been estimated that as many as 50% of foals have ulcers within one month of birth. Stomach ulcers afflict other types of performance horses as well. Only pastured horses seem to be relatively free from gastric ulcers.

It is believed that the high incidence of ulcers in horses is attributable to the steady secretion of hydrochloric acid and pepsin in the horse's stomach as part of its normal digestive process, perhaps exacerbated by other factors. The cells of the stomach contain acid-stimulating receptors. These receptors control an enzyme system that causes acid to be pumped into the stomach of the horse 24 hours a day. Excess acid is thought to contribute to ulcer formation, although other factors, such as a restricted diet and training are also thought to be factors.

Unfortunately, there are generally no external symptoms of ulcers in horses until the condition is advanced. By then, performance may be suffering and appetite might be reduced. Because of the value of race horses, preventative treatment is often recommended to avoid or control ulcers.

Three types of medications are given to horses to treat them for gastric ulcers: “H2 antagonists” (where “H2” means hystamine, type 2), “proton pump inhibitors” and antacids. Typically, H2 antagonist medication is given to reduce or neutralize acid in the horse's stomach. Cimetidine, ranitidine, famotidine and nizatidine are examples of H2 antagonist medications.

The so-called proton pump inhibitor act to inhibit secretion of acids. One particular proton pump inhibitor given to horses to prevent or treat ulcers is omeprazole, sold under the trademark GASTROGARD and manufactured by AstraZeneca Group. Omeprazole belongs to a class of anti-secretory compounds, the substituted benzimidazoles. (Pantoprozole is also a substituted benzimidazole.) Specifically, omeprazole is 5-methoxy-2-[[(4-methoxy-3,5-dimethyl-2-pyridinyl)methyl]sulfinyl]-1-H-benzimidazole. Empirically, this compound is C₁₇H₁₉N₃S. This compound, sold as a flavored paste containing 37% w/w omeprazole for administration through an oral syringe or a nasogastric tube in doses of up to 1.5 mg/kg per day, suppresses acid production by inhibiting the stomach's acid-stimulating receptors.

Antacids, commonly used by humans, have also been examined for use in horses to some extent. Typically, these contain salts such as calcium carbonate, aluminum hydroxide or magnesium hydroxide. For example, a typical antacid sold without prescription contains 7.2 g magnesium hydroxide/8.1 g aluminum hydroxide. These have been successful, but only for a short duration, typically well under an hour after administration. Furthermore, these products must be given in large volumes, approximately ¼ liter four to six times daily. Some antacids have been FDA approved for use in animals. In particular, bismuth salicylate is used to treat vomiting and diarrhea caused by dietary indiscretion, as it soothes the stomach and intestinal lining.

Because of the widespread occurrence of gastric ulcers in horses and the impact they have especially in performance horses, there remains a need for more effective treatments and remedies for equine gastric ulcers.

### SUMMARY OF THE INVENTION

According to its major aspects and briefly recited, the present invention is a composition for treating equine gastric ulcers. The composition, intended for oral administration, comprises a mixture of an acid antagonist, bismuth salicylate, and glycerin, mixed with a flavored sweetener. Preferably, the acid antagonist is a proton pump inhibitor and most preferably omeprazole and the ratio of acid antagonist to bismuth salicylate is ranges from 0.28 to 0.85 by weight and a sufficient quantity of glycerin is used to form an oral paste. The composition is given using 15 ccs from an oral syringe per day.

The present composition is more effective than acid suppressor by itself or with a thickener. While not wishing to be bound by theory, it is believed that the bismuth salicylate acts as an antiinflammatory and coating for the stomach resulting in more relief for the horse during healing.

Other features and their advantages will become apparent to those skilled in treating equine gastric ulcers from a careful reading of the following Detailed Description of Preferred Embodiments.

### DETAILED DESCRIPTION OF PREFERRED EMBODIMENTS

The present invention is a composition for treating equine gastric ulcers. The composition includes an acid antagonist, bismuth salicylate, glycerin, and, preferably, a sweetener and/or a flavoring agent.

The acid suppressor is preferably selected from the group consisting of omeprazole, ranitidine, cimetidine, famotidine, and nizatidine, but most preferably omeprazole. Bismuth salicylate is added to address inflammation and coat the stomach lining. Both the omeprazole and the bismuth salicylate are provided in powder form. Glycerin is blended with them to form a paste suitable for oral administration. The sweetener is preferably a natural or synthetic sweetener, and the flavoring agent can be any pharmacologically acceptable additive used to modify the taste of a mixture, but is preferably a natural or synthetic sweetener to which apple, cherry or other acceptable flavor has been added. No thickeners are needed.

The ratio of omeprazole to bismuth salicylate can range from 0.28 to 0.85, but is preferably approximately 0.33, and most preferably about 150 to 460; a sufficient quantity of glycerin is added to make a paste. The omeprazole is in the form of a powder with approximately 1.8 grams of active ingredient in 15 cc based on a 1000 pounds of body weight. Bismuth salicylate is also in the form of a powder with 5.5 gms of active ingredient in 15 cc.

The sweetener with flavoring agent is to improve palatability to the horse or at least to mask the taste of the omeprazole.

Most preferably, for a 1000 lb horse, 151.2 grams of omeprazole powder (1.8 gms/5 cc) are mixed with 1050 cc of glycerin to form a first mixture. Once the first mixture is well blended, 462 grams of bismuth salicylate powder (5.5 gms/15 cc) are added very slowly to blend into the first mixture to form a second mixture. Finally, to form the composition, 5 grams of sweetener and 1 cc of apple flavor are blended to make a paste consistency. The paste is next used to fill 30 cc oral dose syringes. The composition in the amounts given yield approximately 42 syringes.

In addition, a quantity of sucralfate, a sulfated polysaccharide, or bismuth citrate, can be added instead of bismuth salicylate. This compound is useful in the treatment of peptic ulcers in humans and is believed to coat the ulcer itself. An effective amount of sucralfate, when added to the other ingredients, essentially in the same proportion as the bismuth salicylate, coats the ulcer and the stomach, for better relief for the animal during the healing process.

Those skilled in the treatment of equine gastric ulcers will be able to make modifications and substitutions to the foregoing preferred embodiments without departing from the spirit and scope of the present invention, defined by the appended claims.

What is claimed is:

1. A composition for treating equine gastric ulcers, comprising:
an anti-secretory compound;
a salt of bismuth; and
glycerin.

2. The composition as recited in claim 1, wherein said anti-secretory compound is a substituted benzimidazole.

3. The composition as recited in claim 1, wherein said anti-secretory compound is selected from the group consisting of omeprazole, pantoprozole, ranitidine, cimetidine, famotidine, and nizatidine.

4. The composition as recited in claim 1, further comprising a sweetener.

5. The composition as recited in claim 1, further comprising a flavoring agent.

6. The composition as recited in claim 1, wherein the ratio of said anti-secretory compound to said salt of bismuth is 1:3.

7. The composition as recited in claim 1, wherein said anti-secretory compound is omeprazole and said salt of bismuth is bismuth salicylate.

8. The composition as recited in claim 1, wherein sufficient glycerin is added to obtain a paste consistency.

9. The composition as recited in claim 1, wherein said salt of bismuth is selected from the group consisting of bismuth salicylate and bismuth citrate.

10. A method for making a composition for treating equine gastric ulcers, comprising the steps of:
blending an anti-secretory compound and glycerin to make a first mixture; and
adding a salt of bismuth to said first mixture to make said composition.

11. The method as recited in claim 10, further comprising the step of adding a sweetener to said composition.

12. The method as recited in claim 10, further comprising the step of adding a flavoring agent to said composition.

13. The method as recited in claim 12, wherein said anti-secretory compound is selected from the group consisting of omeprazole, pantoprozole, ranitidine, cimetidine, famotidine, and nizatidine and wherein said salt of bismuth is selected from the group consisting of bismuth salicylate and bismuth citrate.

14. The method as recited in claim 10, further comprising the step of filling oral dose syringes with said composition.

15. The method as recited in claim 14, further comprising the step of filling said oral dose syringe with a dose of 15 cc of said composition for a 1000 pound animal.

16. The method as recited in claim 10, wherein said blending step further includes blending sucralfate into said first mixture.

17. The method as recited in claim 10, wherein said glycerin is added in a quantity sufficient to form a paste of said first mixture.

18. The method as recited in claim 10, wherein said omeprazole and said bismuth salicylate in said mixture are present in a ratio ranging from 0.28 to 0.85.

19. The method as recited in claim 18, wherein said ratio is approximately 0.33.

20. The method as recited in claim 19, wherein said ratio is approximately 150 to 460.
